(19) United States
(12) Patent Application Publication
(10) Pub. No.: US 2020/0399591 A1
(43) Pub. Date: Dec. 24, 2020

(54) Protein engineered extracellular vesicles

(71) Applicant: Evox Therapeutics Ltd, Oxford (GB)

(72) Inventors: Justin HEAN, Oxford (GB); Per LUNDIN, Oxford (GB); Andre GORGENS, Huddinge (SE)

(21) Appl. No.: 16/763,145

(22) PCT Filed: Nov. 13, 2018
(86) PCT No.: PCT/EP2018/081122

§ 371 Date: May 11, 2020

(30) Foreign Application Priority Data
Nov. 13, 2017 (GB) 1718681.8

(51) Int. Cl. C12N 5/073; A61K 31/4545; A61K 35/28; A61K 35/50; A61K 9/50; C07K 14/705; C07K 14/79

(52) U.S. Cl. CPC C12N 5/0605; A61K 31/4545; A61K 35/28; C07K 14/79; A61K 9/5068; C07K 14/70596; A61K 35/50

(57) ABSTRACT

The present invention relates to extracellular vesicles (EVs) as a novel therapeutic approach to lysosomal storage disorders (LSD). More specifically, the invention relates to the use of various protein engineering strategies for improving loading of hard-to-load LSD-related proteins and targeting of the resultant EVs to tissues and organs of interest.

### TECHNICAL FIELD

The present invention relates to engineered extracellular vesicles (EVs) as a novel therapeutic approach to lysosomal storage disorders (LSD). More specifically, the invention relates to the use of various protein engineering strategies for improving loading of hard-to-load LSD-related proteins and targeting of the resultant EVs to tissues and organs of interest.

### BACKGROUND ART

Lysosomal storage diseases form a significant subgroup of inborn errors of metabolism. It is estimated that LSDs affect 1 in every 5000 live births, however owing to undiagnosed and misdiagnosed cases this figure is thought to be greater. All LSDs are caused by the accumulation of specific macromolecules or monomeric compounds inside the organelles of the endosomal-autophagic-lysosomal system. Beyond the errors in substrate storage, LSDs are typically associated with neurological disorders and pathologies, accounting for a large proportion of cases of the neurodegeneration cases worldwide. The past two decades have seen advances against LSDs, particularly as a result of the development of enzyme replacement therapies. However, there are still several diseases in which the current therapies require further development, or worse yet, do not exist. Niemann-Pick Type C is an example of one such LSD, which is caused by the absence or malfunctioning of the protein NPC-1. The NPC-1 protein is involved in the colocalization and intracellular transport of large water-insoluble molecules, such as cholesterol and glycolipids. Consequently, under disease settings, free cholesterol and glycosphingolipids accumulate in multiple tissues and organs. Niemann Picks disease has a wide clinical spectrum, which can manifest as hepato-, spleno- or hepatosplenomegaly. Typical to the LSDs, the Niemann Picks results in progressive neurological pathologies, resulting in premature death amongst all cases beyond early childhood. There is currently no cure for Niemann Pick type C (NPC), and current treatment regimens entail disease management.

Previous treatment strategies for NPC included drugs to alleviate symptoms, such as anti-epileptics, anticholinergic or anti-depressants. Since then several candidate drugs aimed at correcting different stages of lipid metabolism pathways have been or are currently being evaluated in a clinical setting, including miglustat and hydroxypropyl-β-cyclodextrin (CD). Miglustat is an imino sugar which inhibits the activity of glucosylceramide synthase, thereby decreasing the production glycosphingolipids. Originally miglustat was developed for the treatment of Gaucher's disease, and was approved in Europe for use in NPC patients in 2009. CD is also currently undergoing clinical trials for the treatment of NPC, via either intrathecal or intravenous routes of administration. CD is believed to interact with cholesterol and either remove it from circulation or shift its distribution. Several challenges are associated with all existing therapies for NPC and most importantly they do not adequately address the underlying disease pathology.

Biopharmaceuticals such as protein biologics (of which one important but often insufficient class of drugs are the above-mentioned enzyme replacement therapies) and RNA therapeutics may provide a more efficacious alternative means to treat LSDs. The introduction of ERTs into clinical practice in the early 1990s resulted in a transformative change in how certain LSDs (e.g. Gaucher's disease) are treated, but ERTs still suffer from significant drawbacks, meaning that many LSD patient categories are still heavily underserved. Although many ERTs carry out their therapeutic effect inside target cells the internalization of these protein-based drugs is not particularly efficient. Furthermore, owing to the large size and charge of essentially all protein biologics, ERTs do not cross the blood-brain-barrier and are therefore hindered from entering the central nervous system (CNS), which is key to the treatment of several LSDs as the neurological manifestations of these diseases are very severe. WO2016/044947 describe attempts to utilize exosomes as a therapeutic modality for LSDs, and said application touches on various approaches for therapeutic intervention however with several significant shortcomings in the design strategy. In fact, whilst WO2016/044947 superficially describes modifying exosomes to target the lysosome of target cells by using fusion protein constructs containing (i) a lysosome targeting sequence fused to (ii) an exosomal protein, the application is conspicuously devoid of disclosures relating to the loading and bioactive delivery of the actual therapeutic mRNA and/or protein-based agent. Evidently, although exosomes have been shown to be excellent carriers of various types of biomolecular cargo, their actual utility for in vivo delivery of therapeutic proteins and/or mRNA is not trivial and requires thoughtful vesicular engineering.

### SUMMARY OF THE INVENTION

It is hence an object of the present invention to overcome the above-identified problems associated with engineering of EVs for delivery of protein therapeutics for LSDs. The present invention addresses several of the key aspects of EV-based therapeutics for LSDs, namely packing and loading of complex, protein drug cargo into the EVs; optimization of the pharmacokinetics of the EVs themselves; harnessing of the regenerative effects of the EVs; and bioactive delivery of the drug cargo (in this case lysosomal proteins, e.g. transporters and enzymes) into target cells in vivo.

The present invention achieves this by utilizing novel EV engineering technology to package and load, in a bioactive state and configuration, the complex and often very large lysosomal proteins needed to treat LSDs. Furthermore, the present invention also involves the serendipitous selection and profiling of EVs with particular molecular characteristics from cell sources that provide an optimal balance between suitable pharmacokinetics and regenerative properties, as well as providing EVs comprising additional protein and nucleic acid components with therapeutic activity in various LSDs. The inventors of the present invention have realized that (i) certain unexpected cell sources are superior in producing correctly folded and, therefore, active lysosomal proteins, (ii) the ability to transport such lysosomal proteins into EVs is highly cell type-dependent and this can be improved by using fusion protein constructs between the therapeutic lysosomal proteins and exosomal proteins, (iii) certain EV protein components are key to biological activity of the cargo protein and of the EVs per se, and (iv) EVs deriving from the EV-producing cell sources herein enables bioactive delivery into the correct intracellular compartments. In contrast, the prior art, for instance WO2016/044947, is completely unconcerned with the selection of optimal cell sources for bioactive delivery of lysosomal proteins and said document is also apparently unaware of the importance of fusing EV enrichment polypeptides to the therapeutic lysosomal proteins per se. WO2016/044947 indeed proposes utilizing fusion protein technology, but merely for targeting the lysosomal compartments of target cells. The fusions of WO2016/044947 are based on short peptide sequences which provide for targeting of the exosomes as such to the lysosomes of target cells, but said fusion proteins do not enable bioactive loading of the actual therapeutic lysosomal proteins per se into EVs. Thus, the prior art is clearly unaware of (1) the importance of cell source selection as a means for targeting to the right intracellular locations, (2) the fact that lysosomal proteins are typically not folded correctly and/or are not bioactive if engineered into any given cell source, and (3) the fact that the loading of therapeutic lysosomal proteins into EVs often requires fusion with an EV enrichment polypeptide/domain to achieve any enzymatic and/or transporter activity in the EVs and importantly in target cells.

In a first aspect, the present invention thus relates to EVs obtainable from mesenchymal stromal cells (MSCs), amnion epithelial (AE) cells or placenta-derived (P) cells, wherein the EVs comprise a plurality of polypeptide constructs comprising at least one lysosomal protein. The AE-EVs, the MSC-EVs and the P-EVs as per the present invention may be engineered to contain various polypeptide constructs for optimizing the packaging and loading into EVs, to target the EVs to tissues and/or cell types of interest, and/or to enhance the trafficking of the EVs into their target compartments for treating different LSDs, i.e. typically the lysosomal compartments of target cells.

Some of the key EVs enrichment strategies as per the present invention include utilizing EV proteins and parts of EV proteins such as syntenin, the N-terminal part of syntenin, or, highly surprisingly, CD63, as fusion partners for therapeutic lysosomal proteins. In advantageous embodiments, the present invention provides for EVs delivering highly hard-to-deliver lysosomal transmembrane proteins, such as the NPC1 protein, cystinosin, the CLN proteins and/or the LAMP2 protein.

In a further aspect, the present invention pertains to polypeptide constructs comprising at least one lysosomal protein and at least one EV enrichment polypeptide, and in additional aspects polynucleotide constructs encoding such polypeptide constructs.

More specifically the lysosomal protein of the polypeptide construct of the present invention may be one or more of NPC1, LAMP2, sialin, CIC5, CLN3, cystinosin, or GM2-activator protein. The at least one EV enrichment polypeptide of the polypeptide construct may be syntenin, the N terminal portion of syntenin, and/or CD63.

Furthermore, the polypeptide of the present invention may further comprise at least one targeting peptide and/or polypeptide.

The present invention also pertains to vectors comprising the polynucleotide constructs of the invention wherein the vector is a plasmid, a mini-circle, and/or any other type of substantially circularized polynucleotide; a virus such as an adenovirus, an adeno-associated virus, a lentivirus, and/or a capsid-free virus; a linear DNA and/or RNA polynucleotide; a messenger RNA (mRNA); and/or a modified mRNA.

The present invention also, in yet another aspect, relates to cells comprising such polypeptide and/or polynucleotide constructs. Such cells are preferably MSCs, AEs and/or a placenta-derived cells, and these cells are preferably stably transfected and/or transduced with such polynucleotide constructs, to allow for consistent and reproducible production of EVs endogenously loaded with a plurality of the polypeptide constructs.

In further aspects, the present invention also relates to pharmaceutical compositions comprising a plurality of EVs as per the present invention, and/or polypeptide constructs of the invention and/or polynucleotide constructs of the invention, and/or any type of vectors according to the invention. The pharmaceutical compositions of the present invention may further comprise a pharmaceutically acceptable carrier. The EVs and/or such pharmaceutical compositions are particularly suitable for the treatment of LSDs but may also be useful in treating various diseases where lysosomal function, morphology, etc. is disturbed, for instance Parkinson's disease, Alzheimer's disease, etc.

In yet other aspects, the present invention relates to methods of increasing the amount of a lysosomal polypeptide in lysosomes and/or in any other cellular compartment of a mammal, as well as various inventive methods for treating LSDs in a subject in need thereof. Such methods comprise being able to select, profile, dose and administer EVs to patients in an optimal manner.

In a preferred embodiment the method of increasing the amount of a lysosomal polypeptide in lysosomes and/or in any other cellular compartment of a mammal, comprises administering to the mammal a composition comprising EVs according to the present invention, at least one polypeptide construct according to the present invention, at least one polynucleotide according to the present invention, and/or at least one vector according to the present invention.

In summary, the present invention provides for highly inventive EV engineering technology to package and load, in a bioactive state and configuration, the complex and often very large lysosomal proteins needed to treat LSDs, and selecting optimal regenerative cell sources with adequate cargo-stabilizing and delivery capabilities.

### BRIEF DESCRIPTION OF THE FIGURES

FIG. 1. NPC1 amnion-derived EVs significantly decreased cholesterol levels in target cells by delivering bioactive NPC1 protein in high amounts to the cells.

FIG. 2. Wharton's jelly MSC-derived EVs (WJ-MSC-EVs, obtainable from Wharton's jelly) were genetically engineered with a polynucleotide construct encoding a polypeptide construct comprising the lysosomal protein cystinosin and the EV enrichment protein CD63, showing potent cysteine transport effects in vitro.

FIG. 3. AE-EVs positive for CD44, SSEA4, CD133, CD24 and engineered to comprise a plurality of NPC1-syntenin polypeptide constructs were evaluated in vivo in a double knock-out mouse model of Niemann-Pick disease. The engineered AE-EVs showed a significant therapeutic effect on both liver and CNS pathology.

### DETAILED DESCRIPTION OF THE INVENTION

By using inventive EV engineering technology coupled with selective design and profiling of bioactive EV populations, the present invention addresses several of the key aspects of EV-based therapeutics for LSDs.

For convenience and clarity, certain terms employed herein are collected and described below. Unless otherwise defined, all technical and scientific terms used herein have the same meaning as commonly understood by one of ordinary skill in the art to which this invention belongs.

Where features, aspects, embodiments, or alternatives of the present invention are described in terms of Markush groups, a person skilled in the art will recognize that the invention is also thereby described in terms of any individual member or subgroup of members of the Markush group. The person skilled in the art will further recognize that the invention is also thereby described in terms of any combination of individual members or subgroups of members of Markush groups. Additionally, it should be noted that embodiments and features described in connection with one of aspects and/or embodiments of the present invention also apply mutatis mutandis to all the other aspects and/or embodiments of the invention. For example, the lysosomal proteins described herein e.g. in connection with the EVs comprising such lysosomal proteins are to be understood to be disclosed, relevant, and compatible with all other aspects, teachings and embodiments herein, for instance aspects and/or embodiments relating to the methods for producing EVs comprising such lysosomal proteins or aspects relating to the polypeptide and/or polynucleotide constructs herein. Furthermore, all polypeptides and proteins identified herein can be freely combined in polypeptide constructs using conventional strategies for fusing polypeptides. As a non-limiting example, all lysosomal proteins described herein may be freely combined in any combination with one or more EV enrichment polypeptides. Also, any and all lysosomal proteins herein may be combined with any other lysosomal protein to generate polypeptide, and/or the corresponding polynucleotide, constructs, comprising more than one lysosomal protein. Moreover, any and all features (for instance any and all members of a Markush group) can be freely combined with any and all other features (for instance any and all members of any other Markush group). Additionally, when teachings herein refer to EVs in singular and/or to EVs as discrete natural nanoparticle-like vesicles it should be understood that all such teachings are equally relevant for and applicable to a plurality of EVs and populations of EVs. As a general remark, the lysosomal proteins, the EV enrichment polypeptides, the targeting peptides and/or polypeptides, the EV-producing cell sources, and all other aspects, embodiments, and alternatives in accordance with the present invention may be freely combined in any and all possible combinations without deviating from the scope and the gist of the invention. Furthermore, any polypeptide or polynucleotide or any polypeptide or polynucleotide sequences (amino acid sequences or nucleotide sequences, respectively) of the present invention may deviate considerably from the original polypeptides, polynucleotides and sequences as long as any given molecule retains the ability to carry out the desired technical effect associated therewith. As long as their biological properties are maintained the polypeptide and/or polynucleotide sequences according to the present application may deviate with as much as 50% (calculated using for instance BLAST or ClustalW) as compared to the native sequence, although a sequence identity that is as high as possible is preferable (for instance 60%, 70%, 80%, or e.g. 90% or higher). The combination (fusion) of e.g. at least one lysosomal protein with at least one EV enrichment polypeptide naturally implies that certain segments of the respective polypeptides may be replaced and/or modified and/or that the sequences may be interrupted by insertion of other amino acid stretches, meaning that the deviation from the native sequence may be considerable as long as the key properties (e.g. the native effects of the lysosomal proteins, EV trafficking and enrichment, targeting properties, etc.) are conserved. Similar reasoning thus naturally applies to the polynucleotide sequences encoding for such polypeptides. All accession numbers and SEQ ID NOs mentioned herein in connection with peptides, polypeptides and proteins shall only be seen as examples and for information only, and all peptides, polypeptides and proteins shall be given their ordinary meaning as the skilled person would understand them. Thus, as above-mentioned, the skilled person will also understand that the present invention encompasses not merely the specific SEQ ID NOs and/or accession numbers referred to herein but also variants and derivatives thereof. All accession numbers referred to herein are UniProtKB accession numbers as per the 10 Nov. 2017 version of the database, and all proteins, polypeptides, peptides, nucleotides and polynucleotides mentioned herein are to be construed according to their conventional meaning as understood by a skilled person.

The terms “extracellular vesicle” or “EV” or “exosome” are used interchangeably herein and shall be understood to relate to any type of vesicle that is obtainable from a cell in any form, for instance a microvesicle (e.g. any vesicle shed from the plasma membrane of a cell), an exosome (e.g. any vesicle derived from the endo-lysosomal pathway), an apoptotic body (e.g. obtainable from apoptotic cells), a microparticle (which may be derived from e.g. platelets), an ectosome (derivable from e.g. neutrophils and monocytes in serum), prostatosome (e.g. obtainable from prostate cancer cells), or a cardiosome (e.g. derivable from cardiac cells), etc. Exosomes and microvesicles represent particularly preferable EVs. The sizes of EVs may vary considerably but an EV typically has a nano-sized hydrodynamic radius, i.e. a radius below 1000 nm. Clearly, EVs may be derived from any cell type, both in vivo, ex vivo, and in vitro. However, the present invention is predominantly focused on EVs obtainable from amnion epithelial (AE) cells, from mesenchymal stromal cells (MSCs), and from placenta-derived cells. Furthermore, the term “EV” and/or “exosome” and/or “microvesicle” shall also be understood to relate to extracellular vesicle mimics, cell membrane-based vesicles obtained through for instance membrane extrusion, sonication, or other techniques, etc. It will be clear to the skilled artisan that when describing medical and scientific uses and applications of the EVs, the present invention normally relates to a plurality of EVs, i.e. a population of EVs which may comprise thousands, millions, billions or even trillions of EVs. As can be seen from the experimental section below, EVs may be present in concentrations such as 10⁵, 10⁸, 10¹⁰, 10¹¹, 10¹², 10¹³, 10¹⁴, 10¹⁵, 10¹⁸, 10²⁵, 10³⁰ EVs (often termed “particles”) per unit of volume (for instance per ml), or any other number larger, smaller or anywhere in between. In the same vein, the term “population”, which may e.g. relate to an EV comprising a certain lysosomal protein shall be understood to encompass a plurality of entities constituting such a population. In other words, individual EVs when present in a plurality constitute an EV population. Thus, naturally, the present invention pertains both to individual EVs and populations comprising EVs, as will be clear to the skilled person. The dosages of EVs when applied in vivo may naturally vary considerably depending on the disease to be treated, the administration route, the activity and effects of the lysosomal protein of interest, any targeting moieties present on the EVs, the pharmaceutical formulation, etc.

The terms “EV enrichment polypeptide”, “EV protein”, “EV polypeptide”, “exosomal polypeptide” and “exosomal protein” are used interchangeably herein and shall be understood to relate to any polypeptide that can be utilized to transport a polypeptide construct (which typically comprises, in addition to the EV enrichment protein, a lysosomal protein) to a suitable vesicular structure, i.e. to a suitable EV. More specifically, these terms shall be understood as comprising any polypeptide that enables transporting, trafficking or shuttling of a fusion protein construct to a vesicular structure, such as an EV. Examples of such exosomal polypeptides are for instance CD9, CD53, CD63, CD81, CD54, CD50, FLOT1, FLOT2, CD49d, CD71 (also known as the transferrin receptor) and its endosomal sorting domain, i.e. the transferrin receptor endosomal sorting domain (as a non-limiting example exemplified by SEQ ID NO 3), CD133, CD138 (syndecan-1), CD235a, ALIX, syntenin (as a non-limiting example exemplified by SEQ ID NO 1) (also known as syntenin-1), the N terminal portion of syntenin (as a non-limiting example exemplified by SEQ ID NO 2) Lamp2b, syndecan-2, syndecan-3, syndecan-4, TSPAN8, TSPAN14, CD37, CD82, CD151, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11 b, CD11c, CD18/ITGB2, CD41, CD49b, CD49c, CD49e, CD51, CD61, CD104, Fc receptors, interleukin receptors, immunoglobulins, MHC-I or MHC-II components, CD2, CD3 epsilon, CD3 zeta, CD13, CD18, CD19, CD30, TSG101, CD34, CD36, CD40, CD40L, CD44, CD45, CD45RA, CD47, CD86, CD110, CD111, CD115, CD117, CD125, CD135, CD184, CD200, CD279, CD273, CD274, CD362, COL6A1, AGRN, EGFR, GAPDH, GLUR2, GLUR3, ARRDC1, HLA-DM, HSPG2, L1CAM, LAMB1, LAMC1, LFA-1, LGALS3BP, Mac-1 alpha, Mac-1 beta, MFGE8, SLIT2, STX3, TCRA, TCRB, TCRD, TCRG, VTI1A, VTI1B, other exosomal polypeptides, and any combinations thereof, but numerous other polypeptides capable of transporting a polypeptide construct to an EV are comprised within the scope of the present invention. Typically, in many embodiments of the present invention, at least one EV enrichment polypeptide is comprised in the polypeptide construct comprising the lysosomal protein, and this fusion polypeptide construct may advantageously also comprise various other components, including linkers, transmembrane domains, cytosolic domains, multimerization domains, release domains, etc.

The terms “source cell” or “EV source cell” or “parental cell” or “cell source” or “EV-producing cell” or any other similar terminology shall be understood to relate to any type of cell that is capable of producing EVs under suitable conditions, for instance in suspension culture, in adherent culture or any in other type of culturing system, and/or in vivo, ex vivo and/or in vitro. Source cells as per the present invention may also include cells producing exosomes in vivo, e.g. via delivery of a polynucleotide construct into a subject for subsequent translation and in vivo production of EVs, in e.g. the liver. The most advantageous source cells per the present invention are MSCs, AE cells, and/or placenta-derived cells, all of which are of mammal, most preferably of human, origin. The MSCs may be obtained from e.g. bone marrow, adipose tissue, Wharton's jelly, perinatal tissue (e.g. amnion, amniotic membrane, amniotic fluid, chorion, placenta, umbilical cord, Wharton's jelly), tooth buds, umbilical cord blood, skin tissue, etc. Generally, EVs may be derived from essentially any cell source, be it a primary cell source or an immortalized cell line. The EV source cells may be any embryonic, fetal, and adult somatic stem cell types, including induced pluripotent stem cells (iPSCs) and other stem cells derived by any method, as well as any adult cell source. The source cell may be either allogeneic, autologous, or even xenogeneic in nature to the patient to be treated, i.e. the cells may be from the patient himself or from an unrelated or related, matched or unmatched donor. In certain contexts, allogeneic cells may be preferable from a medical standpoint, as they could provide immuno-modulatory effects that may not be obtainable from autologous cells of a patient suffering from a certain indication.

In a first aspect, the present invention relates to EVs obtainable from MSCs, AE cells or placenta-derived cells, so called MSC-EVs, AE-EVs, and P-EVs. The EVs as per the present invention are furthermore endogenously engineered to comprise a considerable plurality of polypeptide constructs comprising at least one lysosomal protein, in order to enhance their therapeutic activity in various different LSDs. The term “endogenously engineered” means that EV-producing AE, MSC and/or placenta-derived cells are genetically engineered to contain a polynucleotide construct which encodes for a therapeutic lysosomal protein, which is incorporated into the EVs with the aid of the cellular machinery. The choice of EV-producing cell sources is key to achieving population-wide, high-efficiency loading of EVs with the therapeutic lysosomal protein. In contrast, the prior art, for instance WO2016/044947, merely relates to exosomes (from dendritic cells) that are loaded with a GAA enzyme via electroporation-mediated exogenous loading. Exogenous loading of proteins have several disadvantages, including (i) low efficiency, (2) unequal distribution across an entire EV population, and (3) increased risk for protein misfolding, especially since the therapeutic lysosomal proteins are highly sensitive to conformational changes.

The selection of MSC-EVs, AE-EVs, and P-EVs is based on the unexpected finding that EVs from these cell sources are capable of carrying large number of copies of correctly folded lysosomal proteins, with retained therapeutic activity, be it enzymatic activity or transporter activity or any other activity that said therapeutic lysosomal proteins carry out. Also, the inventors have unexpectedly realized that said cell sources (AEs, MSCs, and placenta-derived cells) produce engineered EVs which are actively delivering the lysosomal proteins to their correct location of action in target cells. Without wishing to be bound by any theory, it is surmised that these properties are a result of the high content of heat shock proteins, particularly heat shock 70 kda protein 8 (also known as Hsp70-8, encoded for by the gene HSPA8), found in EVs, in particular in exosomes, from these cell sources and the proteomic fingerprint that said cells exhibit.

The polypeptide construct comprised in the AE-EVs, the MSC-EVs and the P-EVs may in advantageous embodiments be engineered to further comprise at least one EV enrichment polypeptide, in order to drive the internalization into EVs of the lysosomal protein. Such EV enrichment polypeptides may be selected from essentially any EV polypeptide, for instance from the following group of EV enrichment polypeptides: CD9, CD53, CD63, CD81, CD54, CD50, FLOT1, FLOT2, CD49d, CD71, CD133, CD138, CD235a, ALIX, Syntenin-1, Syntenin-2, Lamp2b, TSPAN8, TSPAN14, CD37, CD82, CD151, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11 b, CD11c, CD18/ITGB2, CD41, CD49b, CD49c, CD49e, CD51, CD61, CD104, Fc receptors, interleukin receptors, immunoglobulins, CD2, CD3 epsilon, CD3 zeta, CD13, CD18, CD19, CD30, CD34, CD36, CD40, CD40L, CD44, CD45, CD45RA, CD47, CD86, CD110, CD111, CD115, CD117, CD125, CD135, CD184, CD200, CD279, CD273, CD274, CD362, COL6A1, ARRDC1, AGRN, EGFR, GAPDH, GLUR2, GLUR3, HLA-DM, HSPG2, Hsp70, L1CAM, LAMB1, LAMC1, LFA-1, LGALS3BP, Mac-1 alpha, Mac-1 beta, MFGE8, SLIT2, STX3, TCRA, TCRB, TCRD, TCRG, VTI1A, VTI1B, any derivatives and/or domains thereof, and any combinations thereof.

The inventors have realized that the most advantageous EV enrichment polypeptides are syntenin, various N terminal portions of syntenin, and CD63. This is highly surprising as syntenin is a soluble, intraluminal EV protein, whereas CD63 is a tetraspanin which is present in the EV membrane. The fact that these two distinct classes of EV protein have such a profound effect on the packaging of lysosomal proteins of various types (transmembrane, soluble, etc.) into EVs is a highly serendipitous discovery and seems to be related to the judicious selection of AE, MSC, and placenta-derived cells as the EV-producing cell sources. The N terminal portion of syntenin and syntenin itself are particularly advantageous EV enrichment polypeptides as they are capable of transporting exceptionally large numbers of lysosomal proteins to EVs almost regardless of where it is inserted into the fusion polypeptide construct. Furthermore, it is also capable of transporting membrane-associated, transmembrane and soluble lysosomal proteins to EVs, with maintained bioactivity.

In further embodiments of the present invention, the lysosomal protein is selected from the group comprising alpha-D-mannosidase, N-aspartyl-beta-glucosaminidase, lysosomal acid lipase, cystinosin, lysosomal associated membrane protein-2 (LAMP2), alpha-galactosidase A, acid ceramidase, alpha-fucosidase, cathepsin A, acid beta-glucosidase, beta-galactosidase, beta-hexosaminidase A, beta-hexosaminidase B, GlcNAc-1-phosphotransferase, beta-galactosylceramidase, lysosomal acid lipase, arylsulfatase A, alpha-L-iduronidase, iduronate-2-sulphatase, paran sulphamidase, acetyl alpha-glucosaminidase, acetyl CoA: alpha-glucosaminide-N-acetyltransferase, N-acetyl glucosamine-6-sulfatase, N-acetyl galactosamine-6-sulfatase, hyaluronidase, acetyl galactosamine-4-sulphatase, beta-glucuronidase, alpha-N-acetyl neuraminidase, N-actiylglucosamine-1-phosphotransferase, mucolipin-1, formylglycine-generating enzyme, palmitoyl-protein thioesterase-1, tripeptidyl peptidase I, cysteine string protein, CLN3p, CLN5p, CLN6p, CLN7p, CLN8p, acid sphingomyelinase, NPC 1, NPC 2, acid alpha-glucosidase, cathepsin K, sialin, alpha-N-acetylgalactosaminidase, GM2 activator, lysosomal acid lipase, and derivatives, regions, domains and/any combinations thereof.

In particularly preferred embodiments as per the present invention, the lysosomal protein is a lysosomal membrane protein, preferably NPC1. NPC1 may be engineered and loaded into the AE-EVs, the MSC-EVs and/or the P-EVs in its native state, but may also be loaded into said EVs through the use of fusion polypeptide construct comprising the NPC1 protein and either one or more of syntenin, the N terminal portion of syntenin, and/or CD63. Other preferred embodiments of the present invention include EVs comprising polypeptide constructs comprising either one of more of the lysosomal proteins LAMP2 (for the treatment of e.g. Danon's disease), sialin (for the treatment of e.g. Salla disease, infantile free sialic acid storage disease), CIC5 (X-linked hypercalciuric nephrolithiasis), CLN3 (Batten disease), or cystinosin (cystinosis), and/or GM2-activator protein (GM2 gangliosidosis).

In further embodiments, the EVs as per the present invention are selected to be positive for various protein markers which surprisingly seems to be associated with regenerative and immune-modulatory effects as well as with suitable pharmacokinetics profiles for the treatment of LSDs. The most bioactive EVs are positive for one (but often at least three) of the following polypeptides: CD63, CD81, CD44, SSEA4, CD133, CD24.

In yet further embodiments, the inventors have realised that EVs from the EV sources herein have inherent effect in various LSDs even without genetic engineering. This is surmised to be a result of the presence in the EVs of various heat shock proteins (Hsps) from the different Hsp protein families, for instance Hsp90, Hsp70—the most important protein of said family is heat shock 70 kDa protein 8 (encoded by the gene HSPA8 and also known as Hsp70-8)—and/or Hsp40. However, there are seemingly other factors being important for this inherent activity of unmodified AE-EVs, MSC-EVs, and P-EVs, as similar therapeutic effects have not been observed with EVs from other cell sources.

Heat shock proteins are known to act as chaperones to assist folding of proteins into their proper conformation. The present inventors have described for the first time specific cell types which produce EVs containing particularly high levels of heat shock proteins which, when modified to endogenously produce and load lysosomal proteins into EVs, result in the endogenously produced lysosomal protein being properly folded and thus highly bioactive. A secondary benefit of this strategy is that treatment of a patient with a lysosomal storage disorder with EVs that are natively rich in heat shock proteins—having the posttranslational modifications created by the EV-producing cell itself and being transported to the target cell in the protected environment of the EVs—is thought to also improve the folding of any mutant lysosomal protein already present in the patient cells. This could account for the inherent beneficial effects seen in un-engineered EVs from these cell sources.

Unlike the prior art, for instance WO2016/044947, the choice of EV-producing cell sources is key to achieving population-wide, high-efficiency loading of EVs with correctly folded therapeutic lysosomal protein. In contrast to WO2016/044947, the present inventors have realized that endogenous loading of EVs with therapeutic lysosomal proteins is a superior approach to achieving bioactive delivery of correctly folded and functional therapeutic lysosomal proteins (both lysosomal enzymes and lysosomal transporters). WO2016/044947 mentions that chaperones (such as heat shock proteins) that are artificially, exogenously added to the exosomes after their formation may facilitate treatment, but it is clear from the exogenous loading approach taken in WO2016/044947 that there is no understanding of the role of native heat shock proteins and other native EV components play in the production of EVs with correctly folded proteins, which then localize to the right intracellular compartment with the lysosomal proteins being maintained in the correct folding with the aid of the heat shock proteins.

Thus, in a preferred embodiment, both the EV-producing cells of the present invention and the EVs produced by those cells comprise a polypeptide construct comprising the therapeutic lysosomal protein as well as at least one heat shock protein, preferably heat shock protein 70 kDa protein 8. The fact that, unlike in WO2016/044947, the heat shock proteins are natively present in the EV-producing cells means that these endogenous heat shock proteins are present in the EVs from the time of their biogenesis and thus ensure that the complex and sensitive therapeutic lysosomal proteins are correctly folded from the outset, i.e. prior to incorporation into EVs, and that they maintain the correctly folded conformation during the production of the EVs by the cell, even after secretion of the EVs from the cells as well as during the purification process, which is expected to lengthen the shelf-life of the manufactured product.

Furthermore, the fact that the heat shock proteins themselves are endogenous and therefore properly post-translationally modified by the EV-producing cell yields better chaperone activity and thus likely better therapeutic activity of the lysosomal proteins. Taken together, the fact that the cells of the present invention employ endogenously produced lysosomal protein in combination with the heat shock proteins endogenously present in the selected producer cell types, this results in lysosomal proteins loaded into the EVs which are significantly better folded, and thus more stable, long lived and bioactive, than lysosomal proteins which are exogenously loaded. Furthermore, the endogenously loaded and endogenously chaperoned lysosomal proteins continue to be maintained in this properly folded conformation once delivered to the target cell again prolonging the bioactivity of the therapeutic protein.

Thus, the combination in the present invention of an EV proteomics fingerprint that contribute to intracellular delivery to the correct compartment, the correctly folded therapeutic lysosomal proteins, and the population-wide presence of high numbers of lysosomal proteins represent a unique approach with far-ranging implications in terms of enhanced intracellular delivery of therapeutic proteins with high potency, improved inherent immune-modulatory effects, and reduced cost of goods.

Thus, in one advantageous embodiment, the present invention relates to compositions comprising a population of AE, MSC and/or P-derived EVs, wherein at least 50%, 60%, or 70% of the EVs are positive for a therapeutic lysosomal protein, more preferably wherein at least 75% of the EVs are positive for a therapeutic lysosomal protein, even more preferably wherein at least 90% of the EVs are positive for a therapeutic lysosomal protein, and/or yet even more preferably wherein at least 95% of the EVs are positive for a therapeutic lysosomal protein. Importantly, the therapeutic lysosomal proteins of the present invention are correctly folded, as a result of the endogenous loading of said proteins into EVs comprising heat shock proteins, which help maintain a correct folding of the proteins in question.

Non-limiting examples of highly potent EVs for the treatment of LSDs as per the present invention include:
MSC-EVs or AE-EVs comprising multiple copies of a polypeptide construct comprising a therapeutic lysosomal protein, as well as at least one native heat shock protein, such as heat shock protein 70 kDa protein 8.
AE-EVs comprising a polypeptide construct comprising lysosomal protein-syntenin, lysosomal protein-N terminal portion of syntenin, or lysosomal protein-CD63. A particularly preferred example are AE-EVs comprising a polypeptide construct comprising NPC1-syntenin, NPC1-N terminal portion of syntenin, or NPC1-CD63.
AE-EVs comprising a polypeptide construct comprising NPC1-syntenin, NPC1-N terminal portion of syntenin, or NPC1-CD63 and being positive for at least one of CD63, CD81, CD44, SSEA4, CD133, CD24. Other lysosomal proteins may naturally replace the NPC1 protein when developing therapies for other LSDs.
AE-EVs comprising a polypeptide construct as per above, and further comprising at least one Hsp, preferably either one or more of Hsp40, Hsp70 (preferably Hsp70-8), and/or Hsp90 or derivatives or domains thereof.
AE-EVs and/or MSC-EVs engineered to comprise a polypeptide construct comprising a plurality of any lysosomal protein, preferably a transmembrane lysosomal protein such as LAMP2, sialin, CIC5, CLN3, cystinosin, GM2-activator protein, or NPC1
Native AE-EVs and MSCs (obtainable from e.g. amnion tissue, placental tissue, Wharton's jelly, bone marrow, or adipose tissue) may also be utilized therapeutically in unmodified form, in particular if they comprise at least one Hsp.

Importantly, the engineering strategies of the present invention for optimizing the EV-producing AE, MSC, and placenta-derived cells result in a highly efficient loading of the lysosomal protein(s) into the AE-EVs, MSC-EVs and P-EVs. Typically, each and every EV as per the present invention comprises at least five to ten copies of the polypeptide constructs (and therefore of the lysosomal protein), but more often well above ten copies, for instance around 20-30 copies, or 30-50 copies, or also above 50 copies, for instance around 75 or around 100 copies of the lysosomal protein in question. Clearly, this is highly important for the therapeutic effect and would not be achievable without the purposely selection of optimal engineering strategies, cell sources, and EV profiles, as well as inventive methods for producing and harvesting such EVs.

In a further embodiment, the EVs may further comprise an organ, tissue or cell targeting peptide and/or polypeptide. An example of a targeting peptide which has proven potent in transporting EVs into the brain and the CNS is the rabies virus glycoprotein (RVG) peptide, but other peptides and polypeptides are also within the scope of the present invention. Importantly, the tissue targeting peptide and/or polypeptide may be comprised in the polypeptide construct which also comprises the lysosomal polypeptide (and optionally the EV enrichment polypeptide) and/or may be present as a separate polypeptide construct in the EVs. When the targeting peptide and/or polypeptide is part of a separate polypeptide construct it is preferably fused to an exosomal protein, to ensure efficient loading into the EVs.

In a further aspect, the present invention relates to a polypeptide construct comprising at least one lysosomal protein and at least one EV enrichment polypeptide. As above-mentioned, such polypeptide constructs may comprise any EV protein (i.e. EV enrichment polypeptide) although certain EV enrichment polypeptides such as CD63 and syntenin and regions of syntenin are highly preferable. Furthermore, as above-mentioned, additional elements may be included in the polypeptides constructs as per the present invention, for instance (i) targeting peptides and/or polypeptides, (ii) multimerization domains such as fold-on domains or leucine zipper domains, (iii) linkers and cleavage or release domains, etc. In advantageous embodiments, the polypeptide construct comprises a lysosomal protein such as either one of more of NPC1, LAMP2, sialin, CIC5, CLN3, cystinosin, or GM2-activator protein.

In yet another aspect, the present invention relates to polynucleotide constructs encoding for the polypeptide constructs as per the present invention. Such polynucleotide construct may naturally be expressed in vivo, ex vivo, and/or in vitro, using various vectors. Suitable vectors comprising the polynucleotide constructs as per the present invention include, in yet another aspect: plasmids; mini-circles; any type of substantially circularized polynucleotide; viruses such as adenoviruses, adeno-associated viruses, lentiviruses, and/or capsid-free viruses; linear DNA and/or RNA polynucleotides; messenger RNAs (mRNAs); and/or modified mRNAs.

In yet further aspects, the present invention relates to cells comprising one or more of the polypeptide constructs, the polynucleotide constructs, or the vectors as described herein. Naturally, the EV-producing cells as per the present are preferably mesenchymal stromal cells (MSCs), amnion epithelial cell (AEs) and/or a placenta-derived cells, but any other cells may also be used. EV-producing cells may be present either in vitro, e.g. in cell culture, or in any ex vivo or in vivo system. The cells as per the present invention may optionally be immortalized, to enable sustained production of the EVs.

In preferred embodiments of the present invention the EV-producing MSC, AE or placenta-derived cell comprises at least one polypeptide construct comprising at least one therapeutic lysosomal protein, at least one polynucleotide construct encoding said polypeptide construct and/or at least one vector according to the invention. The cells of the present invention are typically engineered to comprise the polynucleotide construct (which may be present in the form of a vector such as a plasmid, an mRNA, a linear DNA molecule, a virus or a viral genome, etc.), which is expressed by the cellular machinery into the corresponding polypeptide construct and thereby incorporated into the EVs, normally the exosomes and/or the microvesicles, produced by the cells. Thus, the cells normally initially comprise the polynucleotide construct (or a vector comprising said construct) and once the expression and translation of the polypeptide construct is completed the cell would comprise both the polynucleotide and the corresponding polypeptide constructs, which is normally secreted out from the cell via EV-mediated exocytosis, wherein each and very EV comprises a plurality of copies of the polypeptide construct.

The polypeptide construct comprises at least one therapeutic lysosomal protein, and optionally it comprises the therapeutic lysosomal protein combined in one polypeptide construct with at least one EV enrichment polypeptide, for instance CD63, syntenin, syndecan, Alix, or any other EV enrichment polypeptide which can be operably linked to the therapeutic lysosomal protein on both a polynucleotide and a polypeptide level.

The EV-producing MSC, AE or placenta-derived cell of the present invention may preferably comprise: (a) a polynucleotide construct encoding for a polypeptide construct comprising at least one lysosomal protein which is stably inserted into the EV-producing cell; and/or (b) a polypeptide construct comprising at least one lysosomal protein, which, as abovementioned, may optionally further comprise at least one EV enrichment polypeptide. The creation of a stably (genetically) engineered EV-producing cell source is key to consistent and high-yield production of EVs with a reproducible therapeutic effect and with a reproducible identity from a chemistry, manufacturing and control (CMC) standpoint. The stable cells are normally immortalized, using for instance hTERT immortalization, viral immortalization, and/or conditional immortalization strategies. In order to enable EV production at scale, it is preferable that the EV-producing cells stably comprise the polynucleotide construct (preferably in a suitable vector) over a certain number of population doublings (PDLs), preferably at least 20 PDLs, more preferably at least 50 PDLs, even more preferably at least 70 PDLs, yet even more preferably at least 100 or even at least 200 PDLs.

In a preferable aspect of the present invention at least 50% or 60%, preferably at least 70% or 80%, even more preferably 90% or 95% or more of the EVs produced by the EV-producing MSC, AE or placenta-derived cells comprise a polypeptide construct comprising at least one lysosomal protein.

In another preferable aspect of the present invention the EVs produced by the EV-producing MSC, AE or placenta-derived cells comprise at least 10, 20, 30 or 40 copies, preferably at least 50 copies of the lysosomal protein, more preferably at least 70, 80 or 100 copies of the polypeptide construct comprising the lysosomal protein. The high copy number of therapeutic lysosomal protein in the EVs and the population-wide presence of the therapeutic lysosomal protein result from the innovative selection of the cell sources of the present invention but also by the potential inclusion of EV enrichment polypeptides when needed, as well as by the creation of stably engineered cells for continuous EV production.

In another aspect, the EV-producing MSC, AE or placenta-derived cells preferably comprise one or more of the following native proteins: CD63, CD81, CD44, CD49e, CD105, SSEA4, CD133, CD24, and/or heat shock 70 kDa protein 8. Further preferably, the EV-producing MSC, AE or placenta-derived cells comprise the following native proteins: CD63, CD81, and heat shock 70 kDa protein 8. The combination of these three native proteins in both cells and their corresponding EVs (and more generally tetraspanins combined with Hsp70-8) is closely associated with high-yield EV production, correct folding and activity over extended time periods of the therapeutic lysosomal protein, as well as efficient bioactive delivery to the right compartment in target cells.

The native, continuous presence of heat shock proteins, for instance heat shock proteins from the Hsp70 family such as heat shock 70 kDa protein 8, in both cells and their corresponding EVs is important for the folding and activity of all therapeutic lysosomal proteins, but it is of particular importance for transmembrane therapeutic lysosomal proteins, such as NPC1, cystinosin, sialin, LAMP1, LAMP2, LAMP2A, PPT1, TPP1, CLN3, CLN6, CLN8, MFSD8, glucosylceramidase, LIMP2, Lgp85, Flotillin-1, ATP6V1B1, ATP13A2, CLCN7, OSTM1, HGNSAT, LMBRD1, TRPML1, TRPML3, DIRC2, SPPL2A, DIRC2, DNAJC5, and/or CLN5, etc. Without wishing to be bound by any theory, it is surmised that the presence of native heat shock proteins (such as Hsp70-8) in the cells and in the EVs of the cells already upon translation of the lysosomal transmembrane protein is key to generating and subsequently maintaining correct folding of the protein. Subsequent exogenous addition of heat shock proteins or other chaperones, such as small molecule chaperones, would not be able to salvage an already misfolded protein, which would thus have significant negative ramifications for the activity of the therapeutic lysosomal transmembrane protein in question. Hence the selection in the present invention of EV-producing cell sources with a high level of native heat shock proteins both in the cells and in the corresponding EVs produced by said cells.

In a further aspect, the present invention further relates to a pharmaceutical composition comprising a plurality of EVs as described herein, at least one polypeptide construct, at least one polynucleotide, and/or at least one vector, and a pharmaceutically acceptable carrier. Importantly, all of the biological components herein (EVs, polypeptides, polynucleotide, vectors, cells, etc.) may advantageously be included in a pharmaceutical composition, either alone or together in any combination. Normally, the pharmaceutical compositions of the present invention comprise a population of EVs and a suitable pharmaceutical carrier, additive, and/or excipient.

In another embodiment, the pharmaceutical compositions may advantageously further comprise pharmaceutical agents such as miglustat, arimoclomol, heparin, trehalose, and/or cyclodextrin and/or any derivatives thereof. These types of combinations may result in highly synergistic therapeutic effect, as the EVs deliver a functional lysosomal protein which effect is then potentiated by such pharmaceutical agents. Interestingly, the present inventors have also realized that the miglustat, arimoclomol, and cyclodextrin can be added to the culture of the EV-producing cells, resulting in endogenous loading of said agents into the EVs and enhanced therapeutic effect. This is a very facile method for loading the drugs into the EVs and essentially comprises culturing the EV-producing AE, MSC, or placenta-derived cells in the presence of a suitable concentration of the drug in question. Naturally, the pharmaceutical compositions of the present invention are particularly suited for treating lysosomal storage disorders but other diseases with lysosomal involvement may also be treated using the inventions herein. Such diseases include Parkinson's disease, Parkinson's with GBA, Alzheimer's disease, Crohn's disease, etc.

LSDs of particular interest for treatment using the EVs as per the present invention include the following non-limiting list of diseases: Alpha-mannosidosis, Beta-mannosidosis, Aspartylglucosaminuria, Cholesteryl Ester Storage Disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis Type I, GM1 Gangliosidosis Type II, GM1 Gangliosidosis Type III, GM2—Sandhoff disease, GM2—Tay-Sachs disease, GM2—Gangliosidosis, AB variant, Mucolipidosis II, Krabbe Disease, Lysosomal acid lipase deficiency, Metachromatic Leukodystrophy, MPS I—Hurler Syndrome, MPS I—Scheie Syndrome, MPS I Hurler-Scheie Syndrome, MPS II—Hunter Syndrome, MPS IIIA—Sanfilippo Syndrome Type A, MPS IIIB—Sanfilippo Syndrome Type B, MPS IIIB—Sanfilippo Syndrome Type C, MPS IIIB—Sanfilippo Syndrome Type D, MPS IV—Morquio Type A, MPS IV—Morquio Type B, MPS IX—Hyaluronidase Deficiency, MPS VI—Maroteaux-Lamy, MPS VII—Sly Syndrome, Mucolipidosis I—Sialidosis, Mucolipidosis IIIC, Mucolipidosis Type IV, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Neuronal Ceroid Lipofuscinosis T9, Neuronal Ceroid Lipofuscinosis T10, Niemann-Pick Disease Type A, Niemann-Pick Disease Type B, Niemann-Pick Disease Type C, Pompe Disease, Pycnodysostosis, Salla Disease, Schindler Disease and Wolman Disease.

In an additional aspect, the present invention relates to methods of increasing the amount of a lysosomal protein in lysosomes and/or in any other cellular compartment of a mammal, comprising administering to the mammal a composition comprising either one or more of: (i) EVs, (ii) at least one polypeptide construct, (iii) at least one polynucleotide construct, and/or (iv) at least one vector. Furthermore, the present invention also relates to methods of treatment of an LSD in a subject in need thereof, comprising the steps of: (i) providing a pharmaceutical composition comprising a population of EVs as per the present invention and (ii) administering to a patient a dose of at least 10\hat{ }6 EVs per kg of body weight. Importantly, as abovementioned, the therapeutic intervention may alternatively comprise administering to a patient either the polypeptide constructs, the polynucleotide constructs, and/or the vectors comprising such polynucleotide constructs. This can be carried out using various delivery vectors, e.g. lipid nanoparticles or polymeric or peptide-based delivery vectors. The compositions, the EVs, the polynucleotide and/or polypeptide constructs may be administered to the subject via various administration routes, for instance subcutaneous administration, intravascular and/or intravenous administration, per os administration, intraperitoneal administration, intrathecal administration, intracerebroventricular administration, etc.

### EXPERIMENTAL AND EXAMPLES

### Materials and Methods

Construct design and cloning: Various polypeptide constructs comprising at least one lysosomal protein and optionally other polypeptide domains (such as EV enrichment polypeptides) have been constructed, cloned into vectors and produced in several different EV-producing cell sources, in particular AE cells, MSCs and placenta-derived cells. ORFs were typically generated by synthesis and cloned into the mammalian expression vector pSF-CAG-Amp. Briefly, synthesized DNA and vector plasmid were digested with enzymes NotI and SalI as per manufacturers instruction (NEB). Restricted, purified DNA fragments were ligated together using T4 ligase as per manufacturers instruction (NEB). Successful ligation events were selected for by bacterial transformation on ampicillin-supplemented plates. Plasmid for transfection was generated by ‘maxi-prep’, as per manufacturers instruction.

Cell Culture and Transfection

Depending on the experimental design and assays, in certain cases, non-viral transient transfection and exosome production was carried out in conventional 2D cell culture, whereas in other cases virus-mediated transduction was employed to create stable cell lines, which were typically cultured in bioreactors of different type. Stably engineered EV-producing cells can also be produced using non-viral transfection of the polynucleotide construct of the present invention and/or suitable vectors in which the polynucleotide construct is comprised. For conciseness, only a few examples are mentioned herein.

In the case of viral transduction and viral creation of stable cell lines for various combinations of lysosomal proteins and EV polypeptides, cell sources such as BM-MSCs, WJ-MSC, amnion epithelial cells, amnion mesenchymal stromal cells, or placenta-derived cells were virus-transduced, typically using lentivirus (LV). Typically, 2 uL of LV and optionally Polybrene (or hexadimethrine bromide, final concentration on the well of 8 ug/mL) are added to cell cultures, and 24 hours post transduction the cell medium of transduced cells is changed to fresh complete media. At 72 hours post transduction, puromycin selection (4-6 μg/ml) is performed, normally for 7 days followed by analysis of stable expression of the protein of interest, typically the polypeptide construct comprising the lysosomal protein and optionally other polypeptides such as EV enrichment polypeptides. Stably modified MSCs (for instance from bone marrow, Wharton's jelly, adipose tissue, amnion tissue such as amniotic membrane, amnion fluid, etc.), AE cells or placenta-derived cells were also created using non-viral methods, employing for instance chemical-based transfections and/or physical methods of transfection. Chemical transfection methods comprised introducing a polynucleotide and/or a vector comprising said polynucleotide using e.g. lipid-based agents such as cationic lipids and/or liposomes (often referred to as lipofection), cationic polymers, dendrimers, calcium phosphate, diethylaminoethyl-dextran and/or any combination thereof. Physical methods included electroporation, cell squeezing, microinjection, etc., as well as various other physical methods for creating stable cell lines known to a person skilled in the art. Physical and chemical methods for creating stable cell lines may also be combined, for instance combining lipofection or cationic polymers with electroporation, or any other type of combination for creating stable cells as known to a skilled person.

Stable cells were cultured in either 2D culture or in bioreactors, typically hollow-fibre bioreactors and/or stir-tank bioreactors and/or shaking bioreactors such as Wave bags, and conditioned media was subsequently harvested for exosome preparation. When cells were grown in suspension (e.g. amnion-derived cells) various preparation and purification steps were carried out. The standard workflow comprises the steps of pre-clearing of the supernatant, filtration-based concentration, chromatography-based removal of protein contaminants, and optional formulation of the resultant exosome composition in a suitable buffer for in vitro and/or in vivo assays.

Assays and Analytics

Western blot is a highly convenient analytical method to evaluate the enrichment polypeptide constructs in EVs. Briefly, SDS-PAGE was performed according to manufacturer's instruction (Invitrogen, Novex PAGE 4-12% gels), whereby 1×10¹⁰ exosomes and 20 ug cell lysate were loaded per well. Proteins from the SDS-PAGE gel were transferred to PVDF membrane according to manufacturer's instruction (Immobilon, Invitrogen). Membranes were blocked in Odyssey blocking buffer (Licor) and probed with antibodies against the lysosomal protein and/or the exosomal protein according to supplier's instruction (Primary antibodies—Abcam, Secondary antibodies—Licor). Molecular probes visualized at 680 and 800 nm wavelengths. For EV size determination, nanoparticle tracking analysis (NTA) was performed with a NanoSight instrument equipped with analytical software. For all recordings, a camera level of 13 or 15 and automatic function was used for all post-acquisition settings. Electron microscopy and fluorescence microscopy were frequently used to understand intracellular location and release and to quantitate and analyze EVs.

EVs were isolated and purified using a variety of methods, typically a combination of filtration such as TFF and LC, in particular bead-elute LC and ion-exchange chromatography. Typically, EV-containing media was collected and subjected to a low speed spin at 300 g for 5 minutes, followed by 2000 g spin for 10 minutes to remove larger particles and cell debris. Alternatively, various filters were used for pre-clearing of the supernatant, especially in the case of suspension cells. The supernatant was then filtered with a 0.22 μm syringe filter and subjected to different purification steps. Large volumes were diafiltrated and concentrated to roughly 20 ml using the Vivaflow 50R tangential flow (TFF) device (Sartorius) with 100 kDa cutoff filters or the KR2i TFF system (SpectrumLabs) with 100 or 300 kDa cutoff hollow fibre filters. The preconcentrated medium was subsequently loaded onto the bead-eluate columns (HiScreen or HiTrap Capto Core 700 column, GE Healthcare Life Sciences), connected to an ÄKTAprime plus or ÄKTA Pure 25 chromatography system (GE Healthcare Life Sciences). Flow rate settings for column equilibration, sample loading and column cleaning in place procedure were chosen according to the manufacturer's instructions. The sample was collected according to the UV absorbance chromatogram and concentrated using an Amicon Ultra-15 10 kDa molecular weight cut-off spin-filter (Millipore) to a final volume of 100 μl and stored at −80° C. for further downstream analysis. To assess the protein and RNA elution profiles, media was concentrated and diafiltrated with KR2i TFF system using 100 kDa and 300 kDa hollow fibre filters and a sample analysed on a Tricorn 10/300 Sepharose 4 Fast Flow (S4FF) column (GE Healthcare Life Sciences).

### EXAMPLES

### Example 1

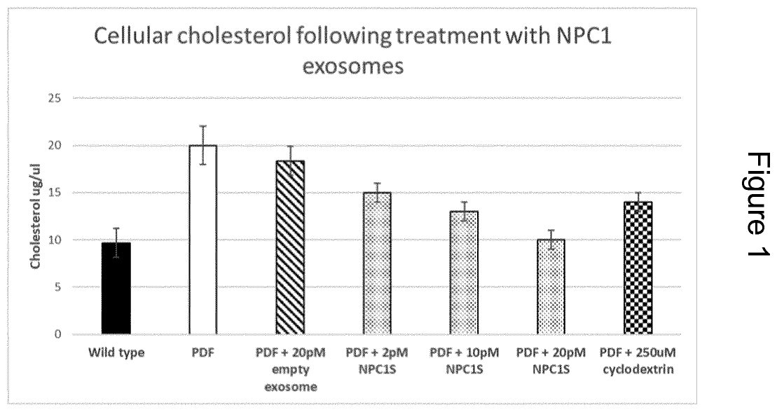

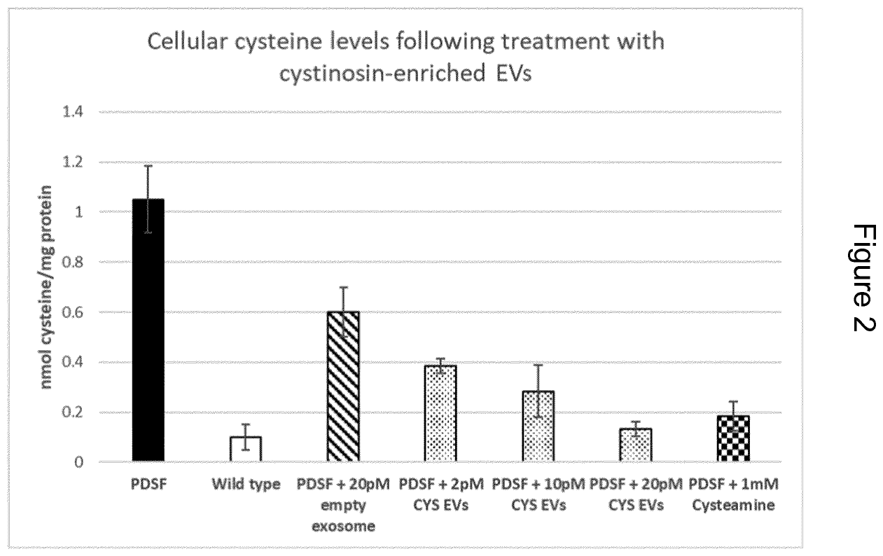

Amnion epithelial (AE) cells, obtainable from post-partum placenta, were genetically engineered (using lentiviral transduction) with a polynucleotide construct encoding a polypeptide construct comprising the NPC1 protein and the N terminal portion of the EV enrichment protein syntenin (NPC1S) (SEQ ID NO 2). AE-EVs produced by the AE cells were harvested, purified, and evaluated in vitro for their ability to enhance cholesterol transport in patient-derived fibroblasts (PDF). As can be seen in FIG. 1, the NPC1 AE-EVs significantly decreased cholesterol levels in the target cells by delivering the bioactive NPC1 protein in high amounts to the cells. Interestingly, when using AE-EVs and MSC-EVs only (and no other cell sources) was it also possible to achieve dose-dependent reduction in cholesterol accumulation by genetically engineering the AE cells to express the NPC1 protein per se, without the presence of any EV enrichment polypeptide (data not shown). The EV-producing cells and their corresponding bioactive EVs were all positive for various heat shock proteins, importantly from the Hsp70 family and more specifically for Hsp70-8.

### Example 2

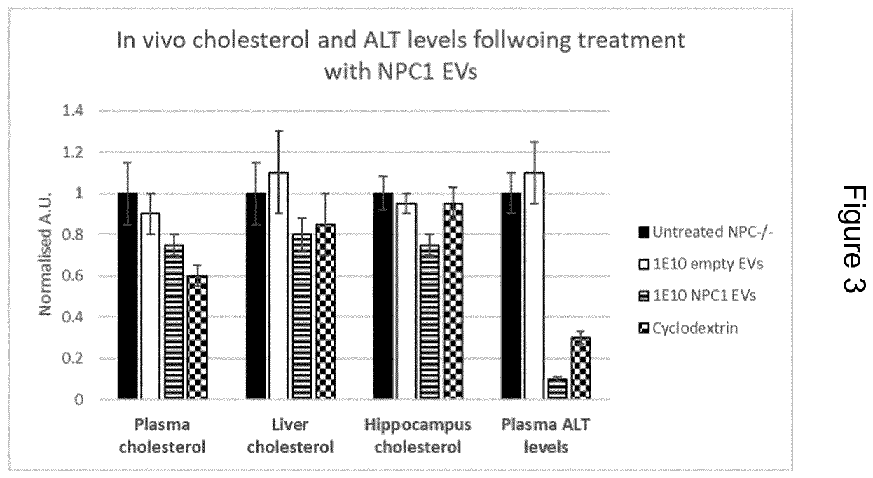

Wharton's jelly MSC-derived EVs (WJ-MSC-EVs, obtainable from Wharton's jelly) were genetically engineered with a polynucleotide construct encoding a polypeptide construct comprising the lysosomal protein cystinosin and the EV enrichment protein CD63. The cystinosin-containing WJ-MSC-EVs (CYS EVs) were harvested from hollow-fibre bioreactor cell culture of the WJ-MSCs, purified, and evaluated in vitro against patient derived skin fibroblasts (PDSF) to assess reduction in cystine accumulation in an in vitro model of cysinosis. FIG. 2 illustrates the strong decrease in cysteine seen when applying the WJ-MSC-EVs comprising the CD63-cystinosin polypeptide construct. As in Example 1, the WJ-MSCs and the WJ-MSC-EVs were positive for Hsp70 proteins, and specifically Hsp70-8, as well as for the tetraspanins CD63 and CD81.

### Example 3

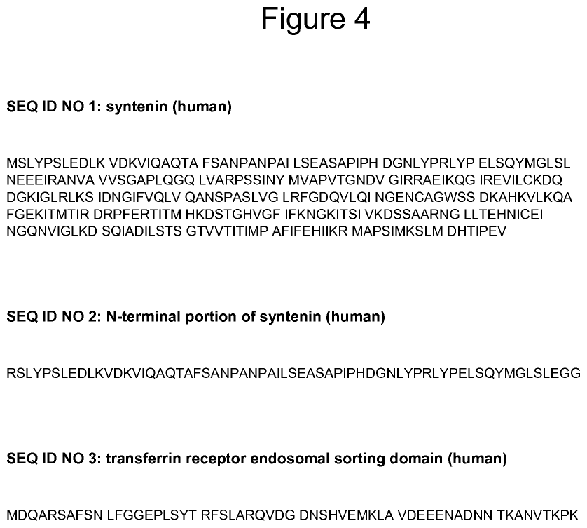

AE-EVs positive for CD44, SSEA4, CD133, CD24, and Hsp70-8 and engineered to comprise a plurality of NPC1-syntenin polypeptide constructs were evaluated in vivo in a double knock-out mouse model of Niemann-Pick disease. The engineered AE-EVs showed a significant therapeutic effect on both liver and CNS pathology (FIG. 3). Non-engineered AE-EVs also showed therapeutic potency, derivable from the high content of various heat shock proteins in the AE-EVs.

1. Extracellular vesicles (EVs) obtainable from mesenchymal stromal cells (MSCs), amnion epithelial (AE) cells or placenta-derived cells, wherein the EVs comprise a plurality of polypeptide constructs comprising at least one lysosomal protein.

2. The EVs according to claim 1, wherein the polypeptide construct further comprises at least one EV enrichment polypeptide.

3. The EVs according to claim 1, wherein the lysosomal protein is selected from the group comprising alpha-D-mannosidase, N-aspartyl-beta-glucosaminidase, lysosomal acid lipase, cystinosin, lysosomal associated membrane protein-2, alpha-galactosidase A, acid ceramidase, alpha-fucosidase, cathepsin A, acid beta-glucosidase, beta-galactosidase, beta-hexosaminidase A, beta-hexosaminidase B, GlcNAc-1-phosphotransferase, beta-galactosylceramidase, lysosomal acid lipase, arylsulfatase A, alpha-L-iduronidase, iduronate-2-sulphatase, paran sulphamidase, acetyl alpha-glucosaminidase, acetyl CoA: alpha-glucosaminide-N-acetyltransferase, N-acetyl glucosamine-6-sulfatase, N-acetyl galactosamine-6-sulfatase, hyaluronidase, acetyl galactosamine-4-sulphatase, beta-glucuronidase, alpha-N-acetyl neuraminidase, N-actiylglucosamine-1-phosphotransferase, mucolipin-1, formylglycine-generating enzyme, palmitoyl-protein thioesterase-1, tripeptidyl peptidase I, cysteine string protein, CLN3p, CLN5p, CLN6p, CLN7p, CLN8p, acid sphingomyelinase, NPC 1, NPC 2, acid alpha-glucosidase, cathepsin K, sialin, alpha-N-acetylgalactosaminidase, GM2 activator, lysosomal acid lipase, and derivatives, regions, domains and/any combinations thereof.

4. The EVs according to claim 1, wherein the EV enrichment polypeptide is selected from the group comprising CD9, CD53, CD63, CD81, CD54, CD50, FLOT1, FLOT2, CD49d, CD71, CD133, CD138, CD235a, ALIX, Syntenin (SEQ ID NO 1), the N terminal portion of syntenin (SEQ ID NO 2), Syntenin-2, Lamp2b, TSPAN8, TSPAN14, CD37, CD82, CD151, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, Fc receptors, interleukin receptors, immunoglobulins, CD2, CD3 epsilon, CD3 zeta, CD13, CD18, CD19, CD30, CD34, CD36, CD40, CD40L, CD44, CD45, CD45RA, CD47, CD86, CD110, CD111, CD115, CD117, CD125, CD135, CD184, CD200, CD279, CD273, CD274, CD362, COL6A1, AGRN, EGFR, GAPDH, GLUR2, GLUR3, HLA-DM, HSPG2, Hsp70, L1CAM, LAMB1, LAMC1, LFA-1, LGALS3BP, Mac-1 alpha, Mac-1 beta, MFGE8, SLIT2, STX3, TCRA, TCRB, TCRD, TCRG, VTI1A, VTI1B, any derivatives and/or domains thereof, and any combinations thereof.

5. The EVs according to claim 2, wherein the EV enrichment polypeptide is syntenin, the N-terminal part of syntenin, or CD63.

6. The EVs according to claim 1, wherein the lysosomal protein is a lysosomal membrane protein.

7. The EVs according to claim 1, wherein the EVs are positive for one or more of the following polypeptides: CD63, CD81, CD44, SSEA4, CD133, CD24, and heat shock 70 kDa protein 8.

8. The EVs according to claim 1, wherein the EVs further comprise at least one heat shock protein.

9. The EVs according to claim 8, wherein the at least one heat shock protein is heat shock 70 kDa protein 8.

10. The EVs according to claim 1, further comprising a tissue targeting peptide and/or polypeptide.

11. The EVs according to claim 10, wherein the tissue targeting peptide and/or polypeptide are comprised in the polypeptide construct and/or is present as a separate polypeptide construct.

12. The EVs according to claim 1, wherein the polypeptide constructs comprising at least one lysosomal protein are substantially correctly folded.

13. The EVs according to claim 1, wherein a single EV comprises:
(i) at least 10 copies of the polypeptide construct;
(ii) at least 50 copies of the polypeptide construct; and/or
(iii) at least 100 copies of the polypeptide construct.

14. A method for producing EVs according to claim 1, comprising the steps of (i) introducing into an EV-producing MSC, AE or placenta-derived cell a polynucleotide construct encoding for a polypeptide construct comprising at least one lysosomal protein, and (ii) obtaining from the EV-producing cell EVs comprising a plurality of polypeptide constructs comprising at least one lysosomal protein.

15. The method according to claim 14, wherein the EVs comprising a plurality of polypeptide constructs comprising at least one lysosomal protein comprise:
(i) at least 10 copies of the lysosomal protein;
(ii) at least 50 copies of the lysosomal protein; and/or
(iii) at least 100 copies of the lysosomal protein.

16. The method according to claim 14, wherein the polypeptide constructs comprising at least one lysosomal protein also comprise at least one EV enrichment polypeptide.

17. An EV-producing MSC, AE or placenta-derived cell comprising at least one polypeptide construct comprising at least one lysosomal protein, at least one polynucleotide construct encoding at least one lysosomal protein, and/or at least one vector comprising a polynucleotide construct encoding at least one lysosomal protein.

18. The EV-producing MSC, AE or placenta-derived cell according to claim 17, wherein a polynucleotide construct encoding for a polypeptide construct comprising at least one lysosomal protein, and/or a vector comprising such a construct, is stably inserted into the EV-producing cell.

19. The EV-producing MSC, AE or placenta-derived cell according to claim 17, wherein the polypeptide constructs comprising at least one lysosomal protein also comprise at least one EV enrichment polypeptide.

20. The EV-producing MSC, AE or placenta-derived cell according to claim 17, wherein:
(i) at least 50%;
(ii) at least 70%; and/or
(iii) at least 90%;
of the EVs produced by the cell comprises the polypeptide construct comprising at least one lysosomal protein.

21. The EV-producing MSC, AE or placenta-derived cell according to claim 17, wherein the EVs produced by the cell comprise:
(i) at least 10 copies of the lysosomal protein;
(ii) at least 50 copies of the lysosomal protein; and/or
(iii) at least 100 copies of the polypeptide construct comprising the lysosomal protein.

22. The EV-producing MSC, AE or placenta-derived cell according to claim 17, wherein the cell is immortalized.

23. The EV-producing MSC, AE or placenta-derived cell according to claim 17, wherein the cells and the EVs produced by the cells comprise one or more of the following native proteins: CD63, CD81, CD44, CD49e, CD105, SSEA4, CD133, CD24, and/or heat shock 70 kDa protein 8.

24. The EV-producing MSC, AE or placenta-derived cell according to claim 17, wherein the cells and the EVs produced by the cells comprise the following native proteins: CD63, CD81, and heat shock 70 kDa protein 8.

25. A pharmaceutical composition comprising a plurality of EVs according to claim 1, and a pharmaceutically acceptable carrier.

26. The pharmaceutical composition according to claim 25, further comprising miglustat, arimoclomol, heparin, trehalose, and/or cyclodextrin and/or derivatives thereof.

27. The EVs according to claim 1, for use in treating one or more lysosomal storage disorder.


SEQUENCE LISTING (3 sequences)

SEQ ID NO: 1 - protein, 298 aa, Homo sapiens
MSLYPSLEDLKVDKVIQAQTAFSANPANPAILSEASAPIPHDGNLYPRLYPELSQYMGLSLNEEEIRANVAVVSGAPLQGQLVARPSSINYMVAPVTGNDVGIRRAEIKQGIREVILCKDQDGKIGLRLKSIDNGIFVQLVQANSPASLVGLRFGDQVLQINGENCAGWSSDKAHKVLKQAFGEKITMTIRDRPFERTITMHKDSTGHVGFIFKNGKITSIVKDSSAARNGLLTEHNICEINGQNVIGLKDSQIADILSTSGTVVTITIMPAFIFEHIIKRMAPSIMKSLMDHTIPEV

SEQ ID NO: 2 - protein, 64 aa, Homo sapiens
RSLYPSLEDLKVDKVIQAQTAFSANPANPAILSEASAPIPHDGNLYPRLYPELSQYMGLSLEGG

SEQ ID NO: 3 - protein, 59 aa, Homo sapiens
MDQARSAFSNLFGGEPLSYTRFSLARQVDGDNSHVEMKLAVDEEENADNNTKANVTKPK